(19) United States
(12) Patent Application Publication
(10) Pub. No.: US 2021/0061286 A1
(43) Pub. Date: Mar. 4, 2021

(54) DRIVING STATE DETERMINATION DEVICE AND DRIVING STATE DETERMINATION METHOD

(71) Applicant: ISUZU MOTORS LIMITED, Tokyo (JP)

(72) Inventor: Mayumi IWAO, Fujisawa-shi, Kanagawa (JP)

(21) Appl. No.: 16/960,850

(22) PCT Filed: Dec. 26, 2018
(86) PCT No.: PCT/JP2018/047785

§ 371 Date: Jul. 8, 2020

(30) Foreign Application Priority Data
Jan. 9, 2018 (JP) 2018-001293

(51) Int. Cl. B60W 40/08; B60W 40/06; B60W 40/114; B60W 40/11; B60W 50/14

(52) U.S. Cl. CPC B60W 40/08; B60W 40/06; B60W 40/114; B60W 40/11; B60W 2540/229; B60W 2552/00; B60W 2520/16; B60W 2520/14; B60W 2540/225; B60W 50/14

(57) ABSTRACT

A control device 10 that determines whether a driver is in a careless driving slate based on the number of times a driver performs a viewing action includes: a number-of-times acquisition unit 33 that acquires the number of times the driver performs viewing actions in a plurality of viewing directions with respect to z traveling direction of a vehicle; a driving state determination unit 36 that determines whether he driver is in the careless driving state based on the number of times of each viewing direction acquired by the number-of-times acquisition unit 33, and a determination value defines by a percentage of viewing action in each viewing direction; a road information acquisition unit 34 that acquires road information on the type of road on which the vehicle travels and a determination value adjustment unit 35 that adjusts the determination value based on the road information acquired by the road information acquisition unit 34.

### TECHNICAL FIELD

The present disclosure relates to a driving state determination device and a driving state determination method

### BACKGROUND ART

In a vehicle such as a truck, there is a concern that a consciousness level of a driver may be lowered and the vehicle may fall into a so-called careless driving state when a monotonous driving state continues. On the ether hand, a device that determines whether the vehicle is in the careless driving slate and issues an alarm when it is determined that the vehicle is in the careless driving state has been proposed (see Patent Literature 1).

When the driver has a high consciousness level, the driver evenly performs viewing actions in a plurality of directions, whereas in the careless driving state, the driver tends to deviate to a viewing action in a specific direction (for example, a lower side viewing action to view a meter panel) Therefore, the above-described device determines that the driver is in the careless driving suite when there is a deviation in a direction in which the driver performs the viewing action.

### CITATION LIST

### Patent Literature

Patent Literature 1: JP-A-2016-095571

### SUMMARY OF INVENTION

### Technical Problem

By the way, depending on the type of road on which the vehicle travels (for example, highways and general roads), even if the driver has a high consciousness level, there is a possibility that the driver does not perform the viewing action evenly in each direction but in a deviated way. In such a case, there is a possibility that the driver may be erroneously determined to be in the careless driving stale even though the consciousness level of the driver is high.

The present disclosure has been made in view of these problems, and an object thereof is to suppress erroneous determination of a careless driving state

### Solution to Problem

In a first aspect of the present disclosure, there is provided a driving state determination device that determines whether s driver is in a careless driving state based on the number of limes the driver performs viewing actions, the driving state determination device including: a number-of-times acquisition unit configured to acquire the number of limes the driver performs viewing actions in a plurality of viewing directions with respect to a direction of a vehicle; a driving state determination unit configured to determine whether the driver is in the careless driving state based on tie number of times in each viewing direction acquired by the number-of-times acquisition unit and a determination value defined by a percentage of the viewing actions in each viewing direction; a road information acquisition unit configured to acquire road information on a type of road on which the vehicle travels; and a determination value adjustment unit configured to adjust the determination value based on the road information acquired by the road information acquisition unit

Further, the determination value adjustment unit may adjust the percentage of the viewing actions in each viewing direction in the determination value based on the road information.

Further, the determination value may include: a first reference value for determining that the driver is not in the careless driving stats; and a second reference value for determining that the driver is in the careless driving state, and the determination value adjustment unit may adjust the first reference value and the second reference value based on the road information.

Further, the number-of-times acquisition unit may acquire the number of times of viewing actions in a yaw direction and a pitch direction with respect to the traveling direction, and in a case where the road information indicates that the road is a first road, the determination value adjustment unit may adjusts such that the percentage of the viewing actions in the yaw direction in the first reference value is deviated compared with a case where the road information indicates that the road is a second road different from the first road.

In a second aspect of the present disclosure, there is provided a driving state determination method for determining whether a driver is in a careless driving state based on the number of times the driver performs viewing actions, the driving state determination method including: a step of acquiring the number of times the driver performs viewing actions in a plurality of viewing directions with respect to a traveling direction of a vehicle; a step of acquiring road information on a type of road on which the vehicle travels; a step of adjusting a determination value defined by a percentage of the viewing actions in each viewing direction based on the acquired road information; and a step of determining whether the driver is in the careless driving suite based on the acquired number of times in each viewing direction and the adjusted determination value.

Advantageous Effects of Invention

According to the present disclosure, it is possible to suppress the erroneous determination of the careless driving suite.

### BRIEF DESCRIPTION OF DRAWINGS

FIG. 1 is a block diagram shoving an example of a configuration of a vehicle 1.

FIG. 2A is a schematic diagram for describing a relationship between a face direction and a viewing action of a driver, and s a schematic diagram showing a relationship between a traveling direction of the vehicle and a pitch direction of the face direction of the driver.

FIG. 2B is a schematic diagram for describing a relationship between the face direction and the viewing action of the driver, and is a schematic diagram showing a relationship between the traveling direction of he vehicle and a yaw direction of the face direction of the driver.

FIG. 3A is a schematic diagram for describing a relationship between a type of road and a determination value, and is a schematic diagram showing a case where the type of road is a highway.

FIG. 3B is a schematic diagram for describing a relationship between a type of road and the determination value, and is a schematic diagram showing a case where the type of road is a general road.

FIG. 4 is a flowchart showing a flow of driving state determination processing.

### CONFIGURATION OF VEHICLE

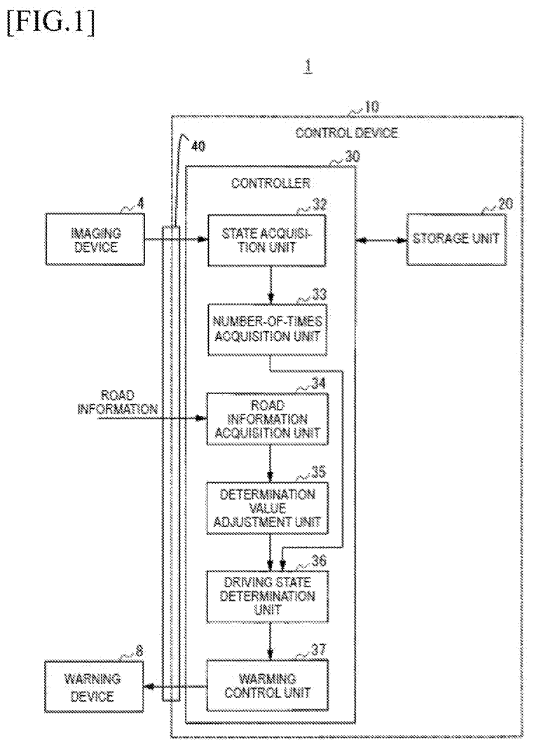

The configuration of a vehicle equipped with a driving slate determination device according to the present disclosure will be described with reference to FIG. 1.

FIG. 1 is a block diagram showing an example of a configuration of a vehicle 1. The vehicle 1 is, for example, a truck. As shown in FIG. 1, the vehicle 1 includes an imaging device 4, an alarm device 8, and a control device 10.

The imaging device 4 is provided in a driver seat (cab of the truck) of the vehicle 1 and includes, for example, a CCD camera. The imaging device 4 captures an image of the driver sitting in the driver seat from a front and generates a captured image. For example, the imaging device 4 captures an image of the driver's face while the vehicle 1 is traveling, and generates a captured image capable of identifying a face direction angle of the driver. The imaging device 4 outputs the generated captured image to the control device 10.

The alarm device 8 is a device that issues an alarm to the driver when the driver is in a careless driving state. When it is determined by the control device 10 that the driver is in the careless driving state, the alarm device 8 issues an alarm. The alarm device 8 includes, for example, a speaker that makes a sound such as an alarm, a display unit (hat displays a warning screen, and a vibration generating unit that generates vibration. Note that the alarm device 8 may issue an alarm by combining at least two of the sound, the display, and the vibration.

The control device 10 controls operations of the imaging device 4 and the alarm device 8. Further, in the present embodiment the control device 10 functions as a driving state determination device that determines whether the driver is in the careless driving state. The careless driving state refers to, for example, a state in which a consciousness level of the driver is lowered following a monotonous driving state on a highway or the like. Specifically, the careless driving state refers to a driving slate in which attention is distracted, such as a state in which the front is not seen carefully, a state of being out of it, and the like. The careless driving state is also a state in which a dozing driving state is a concern.

The control device 10 determines whether the driver is in the careless driving state based on the number of times the driver performs viewing actions in a predetermined direction with respect to a traveling direction or the vehicle (for example, a yaw direction and a pitch direction will) respect to the traveling direction of the vehicle 1). For example, the control device 10 determines that the driver is not in the careless driving slate when the number of limes of viewing actions exceeds a predetermined value within a predetermined time, and determines that the driver is in the careless driving state when the number of times of viewing actions is equal to or less than the predetermined value.

The viewing action is an action for the driver to turn the driver's face in a predetermined direction to confirm safety. The driver usually views the traveling direction of the vehicle 1 via a windshield, but may act, or example, as follows as a viewing action for safety confirmation. Specifically, the driver performs a lower side viewing action for viewing the meter panel of the vehicle 1, a right side viewing action for viewing a right rear side of the vehicle 1 via a right side mirror, and a left side viewing action for viewing a left rear side of the vehicle 1 via a left side mirror. It is known that the viewing action of a side mirror or a meter panel of a driver in a large vehicle such as a truck or a bus is larger in motion and larger in the number of times than a standard-sized passenger vehicle.

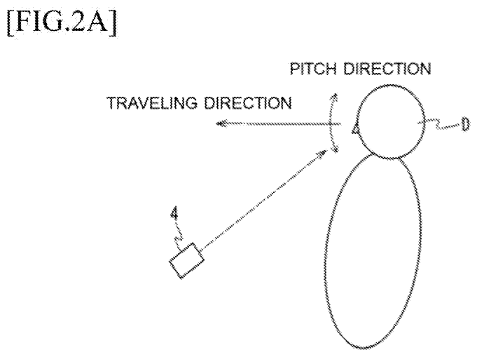

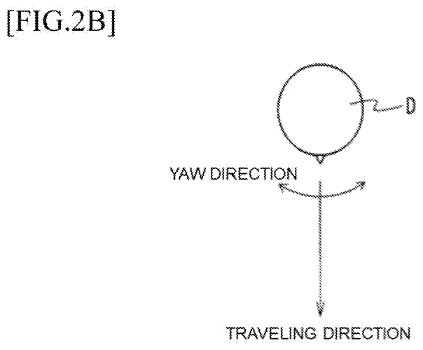

FIGS. 2A and 2B are schematic diagrams for describing a relationship between a face direction and the viewing action of the driver. A driver D turns a face in the pitch direction (FIG. 2A) with respect to the traveling direction of the vehicle 1 and in the yaw direction (FIG. 2B) with respect to the traveling direction to perform viewing. Specifically, the driver D performs the lower side viewing action by turning the face toward a lower side in the pitch direction (negative side shown in FIG. 2A). performs the left side viewing action by turning the face toward a left side in the yaw direction; negative side shown in FIG. 2D), and performs the right side viewing action by turning the face toward a right side in the yaw direction (positive side).

When the driver has a high consciousness level, the driver evenly performs viewing actions in the yaw direction and the pitch direction, whereas in the careless driving state, the driver tends to deviate to a viewing action in a specific direction (for example, the lower side viewing action to view the meter panel). Therefore. there has been proposed a method of determining that the driver is in the careless driving slate when there is a deviation in the direction in which the driver performs the viewing action.

However, depending on the type of road on which the vehicle travels (for example, highways and general roads), even if the driver has a high consciousness level, there is a possibility that the driver does not perform the viewing action evenly in each direction but in a deviated way. In such a case, there is a possibility that the driver may be erroneously determined to be in the careless driving state even though the consciousness level of the driver is high.

In contrast, in the present embodiment, although details will be described later, a determination value defined by a percentage of the viewing actions in each viewing direction is adjusted based on road information on the type of road on which the vehicle travels, the determination value serving as a reference when determining whether the driver is in the careless driving state. Accordingly, even if the viewing action is deviated in a specific direction depending on the road on which the vehicle travels, it is possible to suppress erroneous determination that the driver is in the careless driving state.

<Detailed Configuration of Control Device>

A detailed configuration of the control device 10 as the driving slate determination device will be described with reference to FIG. 1. As shown in FIG. 1. the control device 10 includes a storage unit 20, a controller 30, and in input and output interface 40.

The storage unit 20 is, for example, a Read Only Memory (ROM) or a Random Access Memory (RAM). The storage unit 20stores a program to be executed by the controller 30 and various types of data. The storage unit 20 also stores information such as a threshold used for driving state determination processing.

The controller 30 is, for example, a processor such as a central processing unit (CPU). The controller 30 controls the driving state determination processing by executing the program stored in the storage unit 20. As shown in FIG. 1. the controller 30 includes a state acquisition unit 32, a number-of-times acquisition unit 33, a road information acquisition unit 34, a determination value adjustment unit 35, a driving state determination unit 36, and an alarm control unit 37.

The controller 30 may be configured by. for example, a hardware circuit such as an ASIC.

The input and output interface 40 is communicably connected to the imaging device 4 via a wireless connection or a wired connection, receives a video signal output from the imaging device 4, and inputs the video signal to the controller 30. Further, the input and output interface 40 is communicably connected to an external device such as a navigation system installed in the vehicle or an external device such as a traveling state monitoring system installed in the vehicle by a wireless connection or a priority connection, receives information output from the device such as the navigation system or the traveling state monitoring system, and inputs the information to the controller 30. The input and output interface 40 is communicably connected to the alarm device 8 via a wireless connection or a wired connection, and outputs a signal for controlling the alarm device 8 to the alarm device 8 based on a command output from the controller 30.

The state acquisition unit 32 acquires a face direction state of the driver. The state acquisition unit 32 acquires the face direction state of the driver in the yaw direction and the pitch direction of the driver based on the captured image obtained by capturing the driver's face by the imaging device 4 received via the input and output interface 40. Specifically, the state acquisition unit 32 acquires the face direction angle, which is an angle when the driver turns the face from the traveling direction to the yaw direction and the pitch direction.

The number-of-times acquisition unit 33 acquires the number of times the driver performs viewing actions in a plurality of viewing directions. The number-of-times acquisition unit 33 acquires the number of times of the viewing actions in the yaw direction and the pitch direction with respect to the traveling direction of the vehicle as the plurality of viewing directions. Few example, when the value (face direction change amount) of the face direction angle in the yaw direction is larger than the threshold, the number-of-times acquisition unit 33 counts this action as a viewing action in the yaw direction, and when the value of the face direction angle in the pitch direction exceeds the threshold, the number-of-times acquisition unit 33 counts this action as a viewing action in the pitch direction. However, the present disclosure is not limited to the above, and instead of the face direction angle, when an angular velocity at the time of the face direction change in the yaw direction and the pitch direction exceeds a threshold, this action may be counted as the viewing action.

The road information acquisition unit 34 acquires road information on the type of road on which the vehicle 1 travels based on tie information received via the input and output interface 40. The road information acquisition unit 34 may determine, for example, the type of road on which the vehicle 1 is traveling from the map information. Examples of the type of road include a highway and a general road. The road information may include information on a lane of the road on which the vehicle 1 is traveling.

The determination value adjustment unit 35 adjusts the determination value based on the road information acquired by the road information acquisition unit 34. The determination value is for determining whether the driver is in the careless driving state when the viewing action is deviated in the specific direction. For example, the determination value is defined by a percentage of the number of times of the viewing action in the yaw direction to the number of times of the viewing action in the pitch direction.

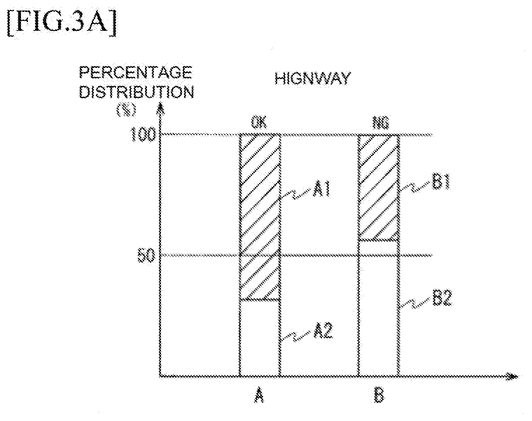

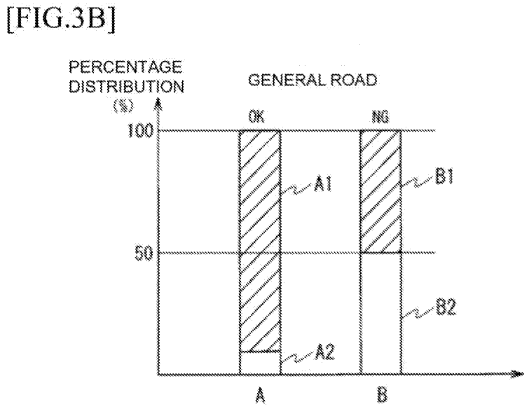

FIGS. 3A and 3B are schematic diagrams for describing a relationship between the type of road and the determination value. The determination value includes a first reference value A serving as a reference for determining that the driver is not in the careless driving state, and a second reference value B serving as a reference for determining that the driver is in the careless driving state. FIG. 3A shows tie first reference value A and the second reference value B when the road is a highway, and FIG. 3B shows the first reference value A and live second reference value B w hen the road is a general road. The first reference value A includes a yaw percentage A1 indicating a percentage of the viewing action in the yaw-direction (here, the left side viewing action and the right side viewing action), and a pitch percentage A2 indicating a percentage of the viewing actions in the pitch direction (here, the lower side viewing action). The second reference value B includes a yaw percentage B1 indicating a percentage of the viewing actions in the yaw direction and a pitch percentage B2 indicating a percentage of the viewing actions in the pitch direction.

The percentage of the left side viewing action and the percentage of the right side viewing action may be further divided for the yaw percentages A1, B1. For example, in the highway of FIG. 3A, the percentage is set to a value that is determined to be the careless driving state when the viewing action is deviated to one of the left side viewing action and the right side viewing action.

The determination value adjustment unit 35 adjusts the first reference value A and the second reference value B based on the road information. For example, the determination value adjustment unit 35 adjusts the reference values to the first reference value A and the second reference value B in FIG. 3A when the load information indicates that the road is a highway, and adjusts the reference values to the first reference value A and the second reference value B in FIG. 3B when the road information indicates that the road is a general road. Specifically, in a case where the road information indicates that the road is a highway (first road), the determination value adjustment unit 35 increases the percentage of the viewing actions in the pitch direction in the first reference value A as compared with the case where the road information indicates that the road is a general road (second road).

The driving state determination unit 36 determines whether the driver is in the careless driving state according to the deviation of the viewing action based on the percentage of the viewing actions in each viewing direction. For example, when the vehicle travels on a highway, although there is a deviation where the number of times of the viewing action in the yaw direction is large (see FIG. 3A). the driving stale determination unit 36 docs not determine that the driver is in the careless driving state even if there is a deviation in the viewing action in the yaw direction.

The driving state determination unit 36 determines whether the driver is in the careless driving state based on the number of times acquired by the number-of-times acquisition unit 33 and the determination value defined by the percentage of the viewing actions in each viewing direction. For example, when the percentage of the viewing action in the yaw direction to that in the pitch direction while traveling on the highway is close to the percentage of the first reference value A (FIG. 3A), the driving state determination unit 36 determines that the driver is not in the careless driving state, and when the percentage of the viewing actions is close to the percentage of the second reference value B (FIG. 3A), the driving state determination unit 36 determines that the driver is in the careless driving state.

The driving state determination unit 36 may determine whether the driving state of the vehicle 1 is the careless driving state based on the number of times of the viewing actions within a predetermined time. For example, when the number of times of the viewing actions acquired by the number-of-times acquisition unit 33 within the predetermined time (for example, a total number of times of the viewing actions in the yaw direction and the pitch direction) is less than or equal to the predetermined value, the driving state determination unit 36 determines that the driver Is in the careless driving state, and when the number of times of the viewing actions is larger than the predetermined value, the driving state determination unit 36 determines that the driver is not in the careless driving state.

In the careless driving state, the alarm control unit 37 outputs a signal for controlling the alarm device 8 via the input and output interface 40 to cause the alarm device H to issue an alarm. When the driving state determination unit 36 determines that the driver is in the careless driving state, the alarm control unit 37 causes the alarm device 8 to issue an alarm so as to warn the driver.

<Driving State Determination Processing>

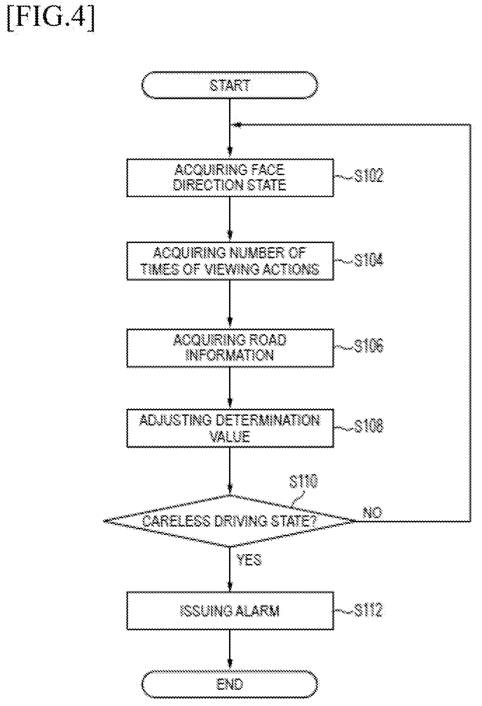

A flow of the driving state determination processing will be described with reference to FIG. 4. The driving state determination processing is realized by executing the program stored in the storage unit 20 by the controller 3) of the control device 10.

FIG. 4 is a flowchart showing the flow of the driving state determination processing. The flowchart of FIG. 4 is started, for example, when the vehicle 1 starts traveling.

First, the slate acquisition unit 32 acquires the face direction state of the driver during driving from the captured image of the imaging device 4 (step S102). Specifically, the state acquisition unit 32 acquires the face direction angle of the driver in the yaw direction and the pitch direction.

Next, the number-of-times acquisition unit 33 acquires the number of times of the viewing actions in the yaw direction and the pitch direction (step S104). For example, the number-of-times acquisition unit 33 obtains the number of times of the viewing action based on the value of the face direction angles in the yaw direction and the pitch direction and the value of the angular velocity when the face direction changes. Accordingly, the percentage of the viewing actions in the yaw direction to that in the pitch direction can be obtained.

Next, the road information acquisition unit 34 acquires road information on the type of road on which the vehicle 1 travels (step S106). For example, the road information acquisition unit 34 acquires the road information indicating a highway or a general road as the type of road.

Next, the determination value adjustment unit 35 adjusts the determination value for determining the careless driving state according to the deviation of the viewing action based on the acquired road information (step S108). For example, the determination value adjustment unit 35 adjusts the reference value to the determination value including the first reference value A and the second reference value B in FIG. 3A when the road information indicates that the road is a highway, and adjusts the reference value to the determination value including the first reference value A and the second reference value B in FIG. 3B when the road information indicates that the road is a general road.

Next, the driving state determination unit 36 determines whether the driver is in the careless driving state based on the adjusted determination value (step S110). For example, when the vehicle 1 is traveling on a highway, the driving state determination unit 36 determines whether the percentage of the viewing actions in the yaw direction to that in the pitch direction is close to one of the first reference value A and the second reference value B in FIG. 3A. When the percentage of the viewing actions in the yaw direction to that in the pitch direction is close to the second reference value B in FIG. 3A. for example, the driving state determination unit 36 determines that the driver is in the careless driving state.

When it is determined in step S110 that the driver is in the careless driving state (Yes), the alarm control unit 37 causes the alarm device 8 to issue an alarm. On the other hand, if it is determined in step S110 that the diver is in the careless driving state (No), the alarm is not issued by the alarm device 8, and the processing of steps S102 to S110 described above is repeated.

<Effect of Present Embodiment>

In the embodiment described above, the control device 10 adjusts a determination value defined by a percentage of the viewing actions in each viewing direction based on the road information on the type of the road on which the vehicle travels, the determination value serving as a reference when determining whether the driver is in the careless driving state.

By adjusting the determination value is described above and determining whether the driver is in die careless driving state by using the adjusted determination value, even if the viewing action is deviated in u specific direction due to the type of road on which the vehicle 1 travels, it is possible to suppress the erroneous determination that the driver is in the careless driving state due to the deviation of the viewing action.

Although the imaging device 4 acquires the face direction actions of the driver in the yaw direction and die pitch direction based on he captured image obtained by capturing the driver's face by the imaging device 4, the present disclosure is not limited thereto. For example, a gyro sensor may acquire the face direction actions of the driver in the yaw direction and the pitch direction. The gyro sensor is attached to a hat or glasses of the driver.

Although the embodiment of the present disclosure has been described above, the technical scope of the present disclosure is not limited to the scope described in the above embodiment. It is apparent to those skilled in the art that various modifications or improvements may he added to the above embodiment. It is also apparent from description of the claims that an embodiment with such modifications or improvements may be included in the technical scope of the present disclosure.

The present application is based on Japanese Patent Application No. 2018-001293 filed on Jan. 9, 2018, contents of which are incorporated herein as reference.

### INDUSTRIAL APPLICABILITY

The present disclosure has an effect of being capable of suppressing erroneous determination of a careless driving state, and is useful to contribute to the realization of a vehicle excellent in safety and traveling performances.

### REFERENCE SIGNS LIST

4 imaging device

10 control device

32 state acquisition unit

33 number-of-times acquisition unit

34 road information acquisition unit

35 determination value adjustment unit

36 driving stale determination unit

1. A driving stale determination device that determines whether a driver is in a careless driving state based on the number of times the driver performs viewing actions, the driving state determination device comprising:
a number-of-times acquisition unit configured to acquire the number of times the driver performs viewing actions in a plurality cf viewing directions with respect to a traveling direction of a vehicle:
a driving slate determination unit configured to determine whether the driver is in the careless driving state based on die number of times in each viewing direction acquired by the number-of-times acquisition unit and a determination value defined by a percentage of the viewing actions in each viewing direction;
a road information acquisition unit configured to acquire road information on a type of road on which the vehicle travels; and
a determination value adjustment unit configured to adjust the determination value based on the road information acquired by the road information acquisition unit.

2. The driving state determination device according to claim 1,
wherein the determination value adjustment unit is configured to adjust the percentage of the viewing actions in each viewing direction in the determination value based on the road information .

3. The driving state determination device according to claim 2,
wherein die determination value includes: a first reference value for determining that the driver is not in the careless driving state; and a second reference value for determining that the driver is in the careless driving state, and
wherein the determination value adjustment unit is configured to adjust the first reference value and the second reference value based on the road information.

4. The driving state determination device according to claim 3,
wherein the number-of-times acquisition unit acquires the number of limes of viewing actions in a yaw direction and a pitch direction with respect to the traveling direction, and
wherein, in a case where the road information indicates that the road is a first road. the determination value adjustment unit adjusts such that the percentage of the viewing actions in the yaw direction in the first reference value is deviated compared with a case where the road information indicates that the road is a second road different from the first road.

5. A driving state determination method for determining whether a driver is in a careless driving suite based on the number of times the driver performs viewing actions, the driving state determination method comprising;
a step of acquiring the number of limes the driver performs viewing actions in a plurality of viewing directions with respect to z traveling direction of a vehicle;
a step of acquiring road information on a type of road on which the vehicle travels;
a step of adjusting a determination value defined by a percentage of the viewing actions in each viewing direction based on the required road information; and
a step of determining whether the driver is in the careless driving state based on the acquired number of limes in each viewing direction and the adjusted determination value.

6. A driving stale determination device that determines whether a driver is in a careless driving state based on the number of times the driver performs viewing actions, the driving state determination device comprising;
a controller; and
an input and output interface communicably connected to an external device via a wired connection or a wireless connection, the input and output interface being configured to input a signal received from the external device to the controller, the input and output interface being configured to output a control signal to the external device based on a command output from the controller,
wherein the controller is configured to perform at least the following processing of;
acquiring the number of times the driver performs viewing actions in a plurality of viewing directions with respect to a traveling direction of a vehicle based on a signal input from the input and output interface.
determining whether the driver is in the careless driving state based on the number of times of the viewing actions in each of the plurality of viewing directions and a determination value defined by a percentage of the viewing actions in each of the plurality of viewing directions;
acquiring road information on a type of road on which the vehicle travels based on a signal input form the input and output interface: and
adjusting the determination value based on the road information.
